(19) United States
(12) Patent Application Publication
(10) Pub. No.: US 2006/0157069 A1
(43) Pub. Date: Jul. 20, 2006

(54) Identification method and computer readable medium

(75) Inventor: Kazuhiko Matsumoto, Minato-ku (JP)

Correspondence Address: PEARNE & GORDON LLP, 1801 EAST 9TH STREET, SUITE 1200, CLEVELAND, OH, 44114-3108, US

(73) Assignee: Ziosoft, Inc., Tokyo (JP)

(21) Appl. No.: 11/233,188

(22) Filed: Sep. 22, 2005

(30) Foreign Application Priority Data
Jan. 19, 2005 (JP) 2005-011253

(51) Int. Cl. A61B 19/00

(52) U.S. Cl. 128/898; 600/300

(57) ABSTRACT

A colon containing liquid, other tissues containing liquid and other tissue containing air are shown in the drawings. When boundary surfaces of liquid are extracted from the image using CT values of the respective objects and gradients thereof, a boundary surface of the liquid in the colon and boundary surfaces of the liquids contained in the other tissues are extracted. Next, horizontal sections are extracted from the boundary surfaces. As a result, the boundary surfaces of the liquids contained in the other tissues can be eliminated, thereby enabling extraction of only a horizontal plane of the liquid in the colon. Thereafter, only the liquid in the colon and air in the colon in contact with the horizontal plane are identified as regions in the colon.

This application claims foreign priority based on Japanese patent application No. 2005-011253, filed Jan. 19, 2005, the contents of which is incorporated herein by reference in its entirety.

### BACKGROUND OF THE INVENTION

1. Field of the Invention

The present invention relates to a method and a computer readable medium for identifying two-layered matter which is in an organ and has fluidity.

2. Description of the Related Art

The medical field is revolutionized by the advent of CT (computed tomography) and MRI (magnetic resonance imaging), which enabled direct observation of the internal structure of a human body with progress in image processing technique using a computer. As a result, medical diagnosis using a tomogram of a living body is widely practiced.

Furthermore, in recent years, volume rendering is used in medical diagnosis as a technique for visualizing a three-dimensional internal structure of a human body which is too complicated to understand with only a tomogram, for example. Volume rendering is a technique by which an image of a three-dimensional structure is rendered from three-dimensional digital data of an object obtained by CT.

In addition, for the purpose of discovering polyps or the like in a colon with use of a CT apparatus, virtual endoscopy on the basis of CT images is conducted in place of endoscopy. Usually, a tomogram of a colon shows materials of three types constituted of colon wall tissue, air, and liquid contents (residues). However, when the residues remain on the colon wall, a condition of the colon wall cannot be observed. Therefore, it is desired to obtain an image where the residues are removed from the colon wall.

The residues are required to be identified for removal. Methods of identifying the residues include a method for extracting a residue region using a plurality of threshold values of CT values. In the method using the CT values, by making use of a fact that voxels having intermediate CT values appear in the vicinity of an interface between different substances, an intermediate region can also be extracted through calculation of gradients of the CT values.

FIGS. 8A and 8B show a cross-sectional view of a colon, and a graph on which CT values of substances inside the colon are plotted. More specifically, FIG. 8A is an image obtained by a single slice of a CT scan with respect to a colon 60. In FIG. 8A, a colon wall 61, air 62 (air is usually injected at a time of a CT scan of a colon), liquid 64 (a lumen of the colon is desirably empty at the time of the CT scan, however, a certain amount of moisture and the like (residues) usually remain), and an intermediate region 63 between air and liquid are shown.

FIG. 8B shows a graph on which CT values corresponding to voxels are plotted on a line along the direction of an arrow 65 in FIG. 8A. As shown in FIG. 8A, CT values corresponding to the colon wall 61 (from y1 to y2 and from y5 to y6) are about −100. CT values corresponding to the air 62 (from y2 to y3) are about −1,000. CT values corresponding to the liquid 64 (from y4 to y5) are about 0.

Thus, a region inside the colon 60 where the substances exist forms a two-layered matter which is made of the air and the residues. The substances can be extracted with use of a plurality of threshold values of the CT values. In addition, for instance, voxels having a CT value −500 appear in the intermediate region 63 (from y3 to y4) between the air and the liquid. Therefore, the intermediate region 63 can be extracted on the basis of the CT values and gradients in a graph of the CT values (for example, refer to C. L. Wyatt et al, “Automatic segmentation of the colon for virtual colonoscopy”, Wake Forest University School of Medicine, 2000; S. Lakare et al, “3D Digital Cleansing Using Segmentation Rays”, State Univ. of NY at Stony Brook, 2000; a published Japanese translation of a PCT application No. 2004-500213; a published Japanese translation of a PCT application No. 2004-522464; and U.S. Pat. No. 6,331,116).

However, in the identification method of the related art, difficulty has been encountered in accurately identifying residues inside a colon from a large amount of volume data obtained by a CT apparatus. For instance, as shown in FIG. 9, difficulty has been encountered in accurately identifying the CT values of only the liquid 64 (i.e., residues) inside the colon 60 from the CT values of the liquid 64 inside the colon 60 and those of liquid 64 inside other tissues 71, 73. The reason for this is that the CT values of the residues (most of the residues are moisture, since solid materials are removed in advance with use of purgatives and the like) are close to those of the other tissues having high moisture content. Accordingly, the residues are difficult to distinguish from other tissues. In addition, organs having air inside include lungs, small intestine, and others. When an air-filled organ is adjacent to a tissue having CT values close to that of water, the residues inside the colon cannot be identified only on the basis of a magnitude and gradient of the CT values.

### SUMMARY OF THE INVENTION

The present invention aims at providing an identification method which enables accurate identification of a region in an organ such as a colon.

In the present invention, a method for observing an organ by image processing comprises identifying an interface between two layers made of different substances respectively, based on a condition of the interface being observed in a horizontal plane.

In the present invention, a method for observing an organ by image processing, the method comprises extracting regions each of which is made of any one of at least two different substances, extracting boundary surfaces of the extracted regions respectively, determining a horizontal plane from the extracted boundary surfaces as an interface between two layers which are inside of the organ and correspond to the different substances respectively, and identifying the two layers based on the horizontal plane.

In the present invention, the method further comprises determining that extracted regions which are continuously in contact with the determined interface belong to either of the two layers. In the present invention the different substances are gas and liquid. In the present invention, the horizontal plane is determined by local regions of the boundary surfaces. The reason for the above is that, in many cases the whole boundary surface contains errors in the peripheral portions thereof, and whether the boundary surface is horizontal can not be determined directly and easily by using the boundary surface as it is. However, this problem can be solved by dividing the boundary surface, and determining whether or not each of the divided boundary surface is horizontal.

In the present invention, the boundary surface orthogonal to a direction of gravity is determined as the horizontal plane. In the present invention, the two layers are identified using volume data. In the present invention, the method is executed by network distributed processing. In the present invention, the method is executed by graphic processing unit.

In the present invention, the method further comprises projecting the organ while removing either one of or both the identified two layers.

In the present invention, a computer readable medium has a program including instructions for permitting a computer to observe an organ by image processing. The instructions comprise extracting regions each of which is made of any one of at least two different substances, extracting boundary surfaces of the extracted regions respectively, determining a horizontal plane from the extracted boundary surfaces as an interface between two layers which is inside of the organ and correspond to the different substances respectively, and identifying the two layers based on the horizontal plane.

According to the invention, by use of a horizontal plane, two regions in contact with the horizontal plane can be identified accurately.

### BRIEF DESCRIPTION OF THE DRAWINGS

FIGS. 1A, 1B and 1C are views for explaining an overview of an identification method of an embodiment of the present invention.

FIG. 2 is a flowchart (1) for explaining the identification method of an embodiment of the present invention.

FIG. 3 is a flowchart (2) for explaining the identification method of an embodiment of the present invention.

FIGS. 4A, 4B, 4C and 4D are views for explaining extraction of a boundary surface according to the identification method of an embodiment of the present invention.

FIGS. 5A, 5B, 5C and 5D are views for explaining smoothing processing according to the identification method of an embodiment of the present invention.

FIGS. 6A and 6B are views for explaining extraction of horizontal sections according to the identification method of an embodiment of the present invention.

FIGS. 7A, 7B and 7C are views for explaining identification of a colon according to the identification method of an embodiment of the present invention.

FIG. 8A shows a cross-sectional view of a colon.

FIG. 8B shows a graph on which CT values of substances in a colon are plotted.

FIG. 9 is a view showing an image obtained by a single slice of a CT scan with respect to a colon and other tissues.

### DESCRIPTION OF THE PRFERRED EMBODIMENTS

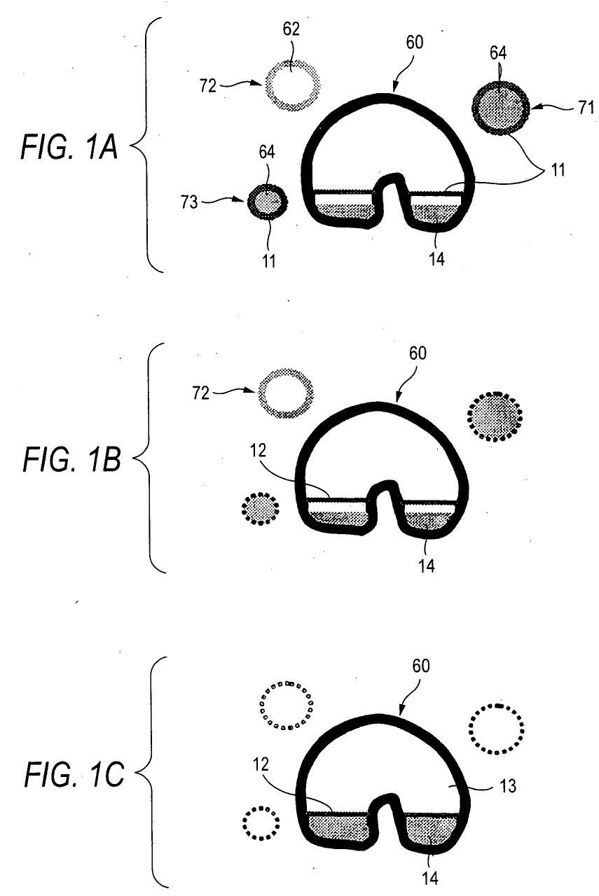

FIGS. 1A to 1C are views for explaining an overview of an identification method for explaining an embodiment of the present invention. FIGS. 1A to 1C show images of a colon and other tissues obtained by a CT scanner. In the identification method of the embodiment, at first, as shown in FIG. 1A, boundary surfaces of liquid are extracted on the basis of voxel values obtained by the CT apparatus. FIG. 1A shows a colon 60 containing in-colon liquid 14, other tissues 71, 73 containing liquids 64, and other tissue 72 containing air 62. When the boundary surfaces of the liquid are extracted from the image by using the CT values of the respective substances and gradients thereof, a boundary surface 11 of the in-colon liquid 14 and boundary surfaces 11 of the liquids 64 contained in the other tissues 71, 73 are extracted.

Next, as shown in FIG. 1B, horizontal sections are extracted from the boundary surfaces 11. As a result of the extraction, the boundary surfaces 11 of the liquids 64 contained in the other tissues 71, 73 can be eliminated, thereby enabling identification of only a horizontal plane 12 of the in-colon liquid 14. Since the residues are mainly constituted of moisture, a horizontal plane is formed between the residues and the air at the time of imaging. In particular, as compared with other planes inside the body, the horizontal plane is placed under the restraint of its orientation by the presence of gravity. Therefore, the orientation of the horizontal plane includes important information. The horizontal plane can be calculated on the basis of gravity information. Accordingly, as shown in FIG. 1C, only the in-colon liquid 14 and in-colon air 13 in contact with the horizontal plane 12 are identified as an in-colon region.

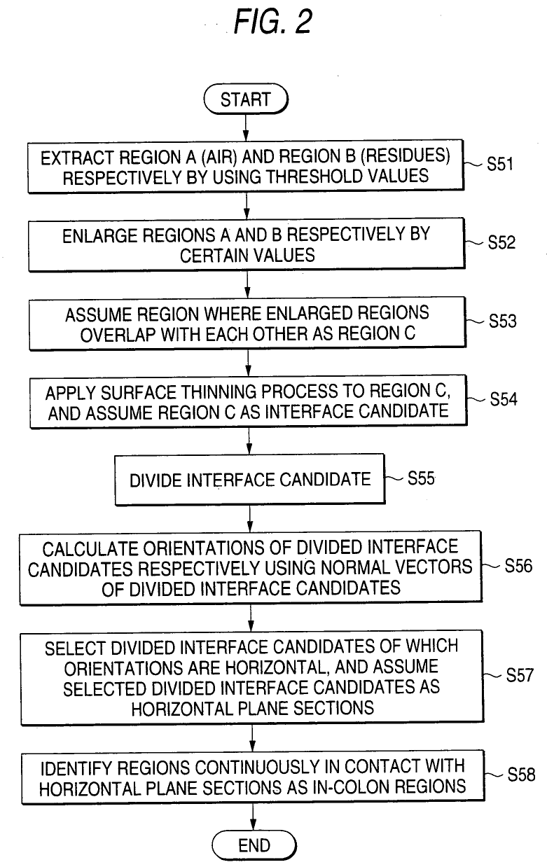

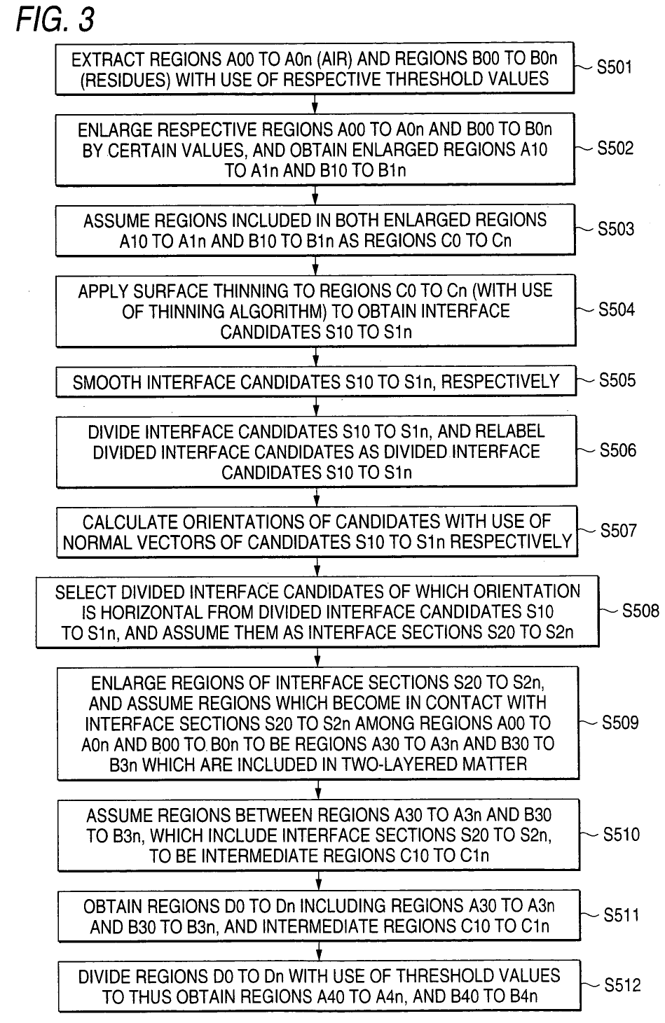

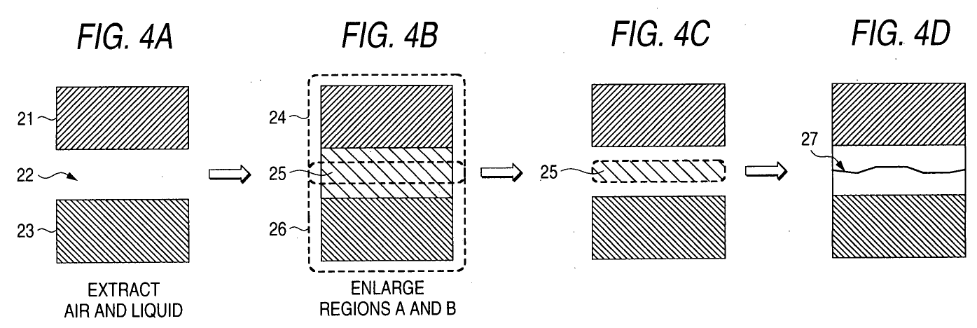

FIGS. 2 and 3 are flowcharts for describing the identification method of the embodiment of the present invention. FIGS. 4A to 4D, 5A to 5D, 6A and 6B, and 7A to 7C are views for explaining extraction of a boundary surface, smoothing processing, extraction of horizontal sections, and identification of a colon, according to the identification method of the embodiment of the present invention. The identification method of the embodiment will be described with reference to the drawings.

In FIG. 4A, air 21 and liquid 23 constitute a two-layered matter having fluidity. In the first step of the identification method of the embodiment, a region A corresponding to the air 21 and a region B corresponding to the liquid 23 are respectively extracted by use of threshold values (step S51 in FIG. 2). In this step, an intermediate region 22 between the air 21 and the liquid 23 is not detected. Next, as shown by dotted lines 24 and 26 in FIG. 4B, the extracted region A of the air 21 and the extracted region B of the liquid 23 are enlarged by certain values, respectively (step S52). As shown in FIG. 4C, a region where the enlarged regions overlap with each other is taken as a boundary region C 25 (step S53).

Next, thinning process (surface thinning) is performed on the boundary region C 25 with use of a thinning algorithm, thereby extracting an interface 27 (interface candidate) as shown in FIG. 4D (step S54). More specifically, thinning process is performed on the boundary region C 25, thereby extracting voxel groups forming the boundary region C 25. The voxel groups are connected to form a polygonal plane. Furthermore, smoothing is applied to the polygonal plane.

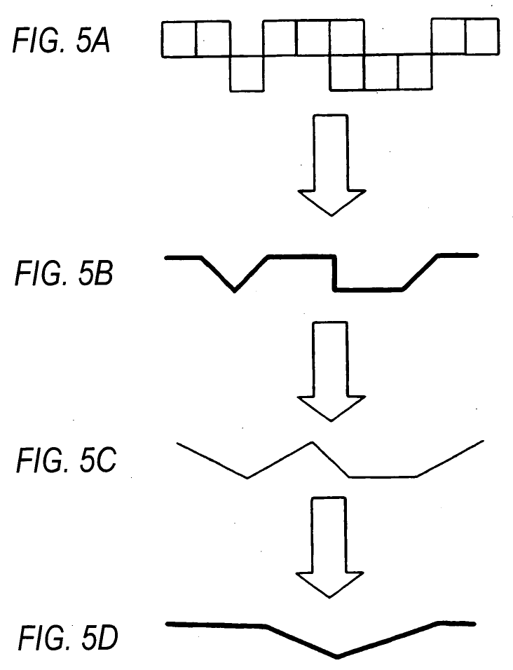

FIGS. 5A to 5D are explanatory views of a flow of smoothing process. From the voxel groups forming the boundary region C 25 as shown in FIG. 5A, a polygonal plane is formed as shown in FIG. 5B, and smoothing is performed thereto as shown in FIG. 5D. Smoothing is performed because, in many cases, the polygonal plane extracted in such a manner as described above includes noise, whereby whether the polygonal plane is horizontal can not be determined directly and easily.

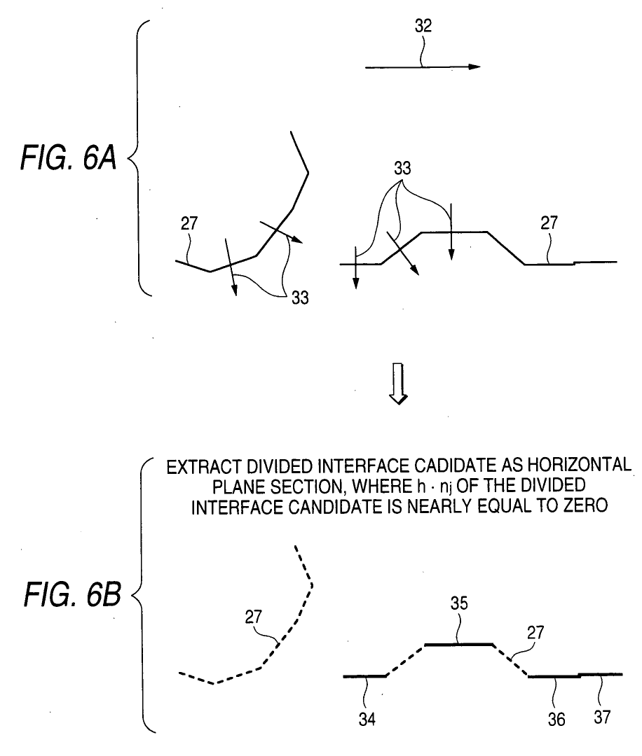

Next, as shown in FIG. 6A, in order to calculate an orientation of the extracted interface candidate 27, the interface candidate 27 is divided into small plane sections, which are referred to as divided interface candidates (step S55). Thereafter, with use of normal vectors of the divided interface candidates, an orientation of each divided interface candidate is calculated (step S56). As shown in FIG. 6B, the divided interface candidates of which orientations are horizontal are selected, and assumed to be horizontal plane sections 34, 35, 36, and 37 (step S57). In other words, determination about horizontality with respect to the boundary region C 25 is partially performed in steps from S54 to S57.

In this case, a horizontal vector 32 h shown in FIG. 6A is, in a medical image, usually written to an image file as a coordinate system used at the time of imaging. Accordingly, the direction of gravity can be obtained from the coordinate, and a horizontal direction can be obtained from the direction of gravity. More specifically, the horizontal vector 32 h is obtained from data attached to an image, and a normal vector 33 (nᵢ: a normal vector of an i^{th} divided interface candidate) of polygonal planes constituting the interface candidate 27 is calculated respectively. Subsequently, an inner product of the horizontal vector h and the normal vector nᵢ of the polygonal planes is calculated respectively, thereby making a determination as to whether or not the horizontal vector h and the normal vector nᵢ are orthogonal to one another. In this case, the i^{th} divided interface candidate is determined as horizontal when ε is assumed to be a certain threshold value which is nearly equal to zero, and when h·nᵢ<ε satisfied. Meanwhile, as the determination is performed for each polygonal plane, there is a possibility that the obtained horizontal plane sections 34, 35, 36, and 37 are fragmented.

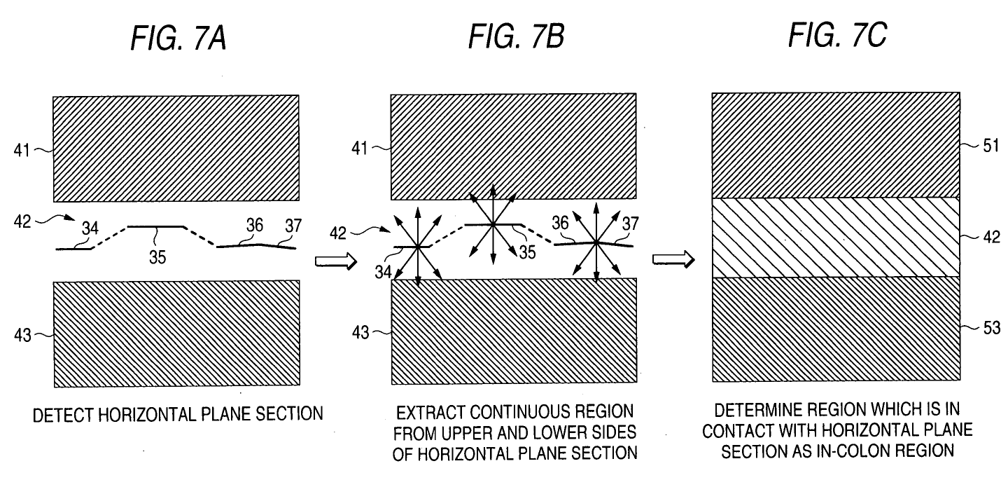

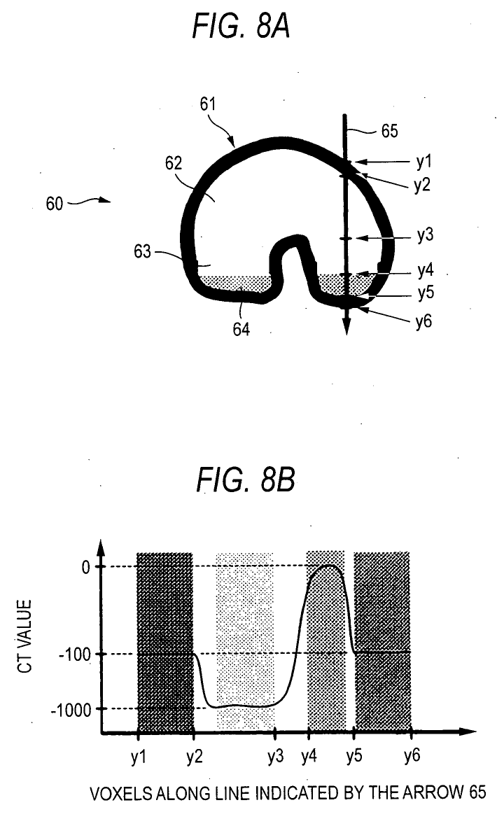

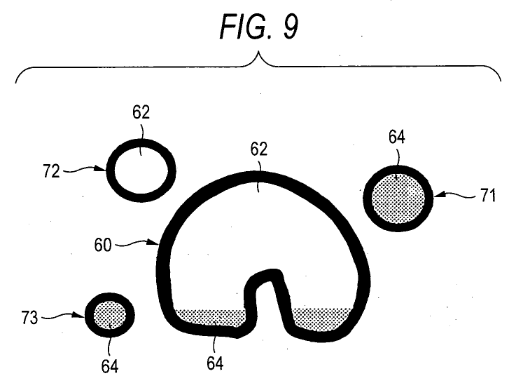

FIG. 7A shows the horizontal plane sections 34, 35, 36, and 37 extracted from an intermediate region 42 between the region A of air 41 and the region B of liquid 43.

Next, upper and lower sides of the horizontal plane sections 34, 35, 36, and 37 are respectively scanned. Subsequently, as shown in FIG. 7B, continuous regions are extracted from the upper and lower sides of the horizontal plane sections 34, 35, 36, and 37. Regions (, in the intermediate region 42 between the air 41 and the liquid 43,) continuously in contact with the horizontal plane sections 34, 35, 36, and 37 are identified as in-colon air 51 or in-colon liquid 53, respectively (FIG. 7C and step S58).

FIG. 3 is a detailed flowchart of the flowchart shown in FIG. 2. First, regions A00 to A0n (air) and regions B00 to B0n (residues) are extracted with use of respective threshold values (step S501). The reason for extracting a plurality of regions of A00 to A0n and a plurality of regions of B00 to B0n is to ensure that the whole of the two-layered matter can be extracted afterward in relation to those regions.

Next, the regions A00 to A0n and the regions B00 to B0n are enlarged by certain values respectively, thereby obtaining regions A10 to A1n and B10 to B1n (step S502). In addition, regions included in both the enlarged regions A10 to A1n and B10 to B1n are assumed to be regions CO to Cn (step S503).

Next, surface thinning is performed on regions CO to Cn (with use of thinning algorithm), thereby obtaining interface candidates S10 to S1n (step S504). Then, the interface candidates S10 to S1n are smoothed respectively (step S505) (FIGS. 5A to 5D). The smoothing is applied for removal of noise caused as a result of reduction in the number of the polygonal planes, and the like.

Next, each of the interface candidates S10 to S1n are divided into small sections to be new divided interface candidates S10 to S1n (step S506). Further, orientations of the divided interface candidates are calculated with use of respective normal vectors of the divided interface candidates S10 to S1n (step S507). Further, divided interface candidates of which orientations are horizontal are selected from the divided interface candidates S10 to S1n, and are assumed to be interface sections S20 to S2n (step S508).

Next, regions of the interface sections S20 to S2n are enlarged. Then, among the regions A00 to A0n and B00 to B0n, regions that become in contact with the interface sections S20 to S2n are assumed to be regions A30 to A3n and B30 to B3n included in the two-layered matter (step S509).

Next, regions between the regions A30 to A3n and B30 to B3n, which include the interface sections S20 to S2n, are assumed to be intermediate regions C10 to C1n (step S510). Further, regions D0 to Dn, which include the regions A30 to A3n and B30 to B3n, and the intermediate regions C10 to C1n, are obtained (step S511). The regions D0 to Dn correspond to whole of the two-layered matter. The regions D0 to Dn are divided with use of threshold values, thereby obtaining regions A40 to A4n and B40 to B4n (step S512). The regions A40 to A4n and B40 to B4n respectively correspond to the regions of the respective layers of the two-layered matter.

As a result, the intermediate region between the two layers of the two-layered matter can be identified. In the related-art method for independently identifying respective regions of a two-layered matter, air and residues in a colon are extracted independently. In this case, boundary surfaces of the respective regions do not necessarily coincide with each other. Therefore, in some cases, a space exists between the respective regions. In other cases, a region where both regions are overlapped appears. In particular, a region between air and residues in the colon has a voxel value similar to that of the surrounding tissue, and it is difficult to extract the region directly. In addition, regions which are recognized to be air and residues exist in large numbers. Therefore, according to the method of the related art, the intermediate region cannot be defined in terms of voxel value or geometry, as relationship between air and residues which are in contact with each other can't be obtained. In the present invention, the whole of the two-layered matter including the intermediate region are extracted by use of the horizontal plane sections; and the whole two-layered matter is divided into two regions according to the horizontal plane sections. Accordingly, the respective regions of the two-layered matter can be identified accurately.

As a result, even when the identified horizontal plane sections 34, 35, 36, and 37 are fragmented, the region in the colon can be identified accurately by means of detecting the continuous regions. In this case, air or liquid outside the colon are eliminated, because such air or liquid are not in contact with a horizontal plane. By virtue of the accurate identification of the two-layered matter in the colon as described above, an image in which residues are removed from a lumen of the colon can be obtained.

Meanwhile, in the identification method of the embodiment, extraction of the respective regions of the two-layered matter is performed with use of threshold values. However, a number of other methods for extracting regions have been proposed, and regions may be extracted in accordance with an arbitrary method; for instances, an Active Contour method, a Level Set method, or a Watershed method, etc.

Meanwhile, although respective regions of the two-layered matter are extracted in the identification method of the embodiment, further processing such as enlarging and shrinking may be applied to the respective regions of the two-layered matter. As parameters used in the extraction usually vary between the regions, the processing is for correcting a deviation which occurs in some cases.

Meanwhile, in the identification method of the embodiment, a region between the two layers of the two-layered matter is assumed to be an intermediate region. However, when a region that overlaps with each of the two layers of the two-layered matter exists, the overlapping region may also be assumed as the intermediate region. This is because, when any of the variety of extraction methods is applied, comparatively large regions may be extracted as the two layers in some cases.

Meanwhile, in the identification method of the embodiment, the whole of the two-layered matter is divided into two parts immediately. However, further processing such as enlarging and shrinking may be applied to the whole of the two-layered matter. This processing is for obtaining more accurate identification result.

Meanwhile, in the identification method of the embodiment, boundary surface is extracted by using the intermediate region of the two-layered matter. However, an isosurface may be extracted, or other methods may be employed.

Meanwhile, in the identification method of the embodiment, the two layers of the two-layered matter are made of gas and liquid, such as gas and residues. However, the two layers of the two-layered matter may be made of one type of liquid and another type of liquid, such as oil and water.

Meanwhile, in the identification method of the embodiment, the fragmented horizontal plane sections are immediately assumed as the horizontal plane sections to be obtained. However, further determination of the horizontal plane sections may be performed by making use of size and shape of the horizontal plane sections, and a positional relationship with an adjacent horizontal plane section. When such a determination is performed, selection of an inaccurate horizontal plane section can be prevented.

Meanwhile, in the identification method of the embodiment, detection of a horizontal plane may be performed based on the image processing technique of the related art, and is not limited to the algorithm described above. In addition, the identification method may be applied to identification of residues in a lumen of another organ, such as the stomach.

In addition, according to the identification method of the embodiment, calculations for volume rendering can be divided into those for predetermined image regions or into those for predetermined regions of volume; and, thereafter, the regions can be superimposed. Accordingly, the calculation can be performed by means of parallel processing, network distributed processing, a dedicated processor, or a combination thereof.

In addition, the image processing method of the embodiment can be performed by means of a GPU (Graphic Processing Unit). A GPU is a processing unit which is particularly designed for specialized use in image processing as compared with a general-purpose CPU, and is usually installed in a computer separately from the CPU.

In addition, according to the image processing method of the embodiment, a two-layered matter is identified. However, rendering may be performed with either one of or both layers of the identified two-layered matter being removed. The removal can be implemented by means of removing the regions from the volume data, or by means of applying masking processing to the regions in the two-layered matter. As a result, rendering of a state where residues are removed can be performed. Therefore, it is effective because diagnosis of a portion which has been hidden by the residues and difficult to observe can be performed. In particular, not only parallel projection, but also perspective projection and cylindrical projection can be employed for rendering. In accordance with the perspective projection, a display image for virtual endoscopy can be generated. By means of removing the residues more effective diagnosis is possible. In accordance with the cylindrical projection, an image in which the colon is exfoliated can be generated, and portions which are likely to be missed in an image of the parallel projection or perspective projection can be observed simultaneously, which is effective for diagnosis. The perspective projection and the cylindrical projection will be described below.

For diagnosis of a colon, doctors observe colons by using endoscopes. For a display using the virtual endoscopy corresponding to the endoscope, the perspective projection is used. However, a large amount of residues included in the field of view of the display using the virtual endoscopy has inhibited sufficient observation. However, failure to notice a diseased part in a display using the virtual endoscopy can be reduced by means of identifying the residues with use of the above-mentioned identification method and removing the residues. Meanwhile, since the cylindrical projection uses a viewpoint along a centerline of a colon, the cylindrical projection is appropriate for overviewing-an internal wall of the colon. However, the presence of residues has inhibited simultaneous observation of the entire circumference of the internal wall of the colon. Here, when the projection is performed with removal of the residues identified by the above-mentioned identification method, the entire circumference of the colon can be observed simultaneously. Accordingly, failure to notice lesion in diagnosis with use of the cylindrical projection can be reduced.

In diagnosis with use of the cylindrical projection or perspective projection, a centerline of a colon is obtained in the related art. This is because the centerline of the colon can be used for setting a position of a viewpoint of a virtual endoscope in the perspective projection, and for setting a center axis of the cylinder in the cylindrical projection. However, the presence of residues inhibited automatic obtaining of the centerline of the colon. Accordingly, the centerline of the colon is set by means of, for instance, employing a center line of an air layer or manually. Here, by means of identifying a two-layered matter with use of the above-mentioned identification method, a centerline of the two-layered matter can be obtained. By virtue of this, the centerline of the colon can be obtained automatically. In addition, use of the centerline of the two-layered matter enables efficient setting of the position of the viewpoint of the virtual endoscope in the perspective projection, and the center axis of the cylinder in the cylindrical projection.

In the image processing method of the embodiment, the horizontal direction is calculated based on a direction where gravity is applied by using coordinate information included in the image file. However, a user may specify the horizontal direction. Alternatively, a program may determine the horizontal direction through image analysis, and the like. Further alternatively, the coordinate information may be obtained from a source other than the image file. This is because the coordinate information is not always included in the image file. Meanwhile, the horizontal direction may be determined independently of the direction of gravity. This is because, in some cases, accurate information with regard to the direction of gravity cannot be obtained, and in other cases, the direction of gravity is tilted due to a movement of a patient during image acquiring.

In addition, in the image processing method of the embodiment, the volume data is obtained by means of the CT apparatus. However, the volume data may be obtained from another image apparatus such as an MRI (magnetic resonance imaging) apparatus or PET (positron-emission tomography scan) apparatus. Alternatively, the volume data may be a combined volume data of a plurality of volume data. Further, the volume data may be volume data generated or modified by means of a program or the like.

It will be apparent to those skilled in the art that various modifications and variations can be made to the described preferred embodiments of the present invention without departing from the spirit or scope of the invention. Thus, it is intended that the present invention cover all modifications and variations of this invention consistent with the scope of the appended claims and their equivalents.

What is claimed is:

1. A method for observing an organ by image processing, said method comprising:
identifying an interface between two layers made of different substances respectively, based on a condition of the interface being observed in a horizontal plane.

2. A method for observing an organ by image processing, said method comprising:
extracting regions each of which is made of any one of at least two different substances;
extracting boundary surfaces of the extracted regions respectively;
determining a horizontal plane from the extracted boundary surfaces as an interface between two layers which are inside of the organ and correspond to the different substances respectively; and
identifying the two layers based on the horizontal plane.

3. The method as claimed in claim 2, further comprising:
determining that extracted regions which are continuously in contact with the determined interface belong to either of the two layers.

4. The method as claimed in claim 2, wherein the different substances are gas and liquid.

5. The method as claimed in claim2, wherein the horizontal plane is determined by local regions of the boundary surfaces.

6. The method as claimed in claim 2, wherein the boundary surface orthogonal to a direction of gravity is determined as the horizontal plane.

7. The method as claimed in claim 2, wherein the two layers are identified using volume data.

8. The method as claimed in claim 2, wherein the method is executed by network distributed processing.

9. The method as claimed in claim 2, wherein the method is executed by graphic processing unit.

10. The method as claimed in claim 2, further comprising:
projecting the organ while removing either one of or both the identified two layers.

11. A computer readable medium having a program including instructions for permitting a computer to observe an organ by image processing, said instructions comprising:
extracting regions each of which is made of any one of at least two different substances;
extracting boundary surfaces of the extracted regions respectively;
determining a horizontal plane from the extracted boundary surfaces as an interface between two layers which are inside of the organ and correspond to the different substances respectively; and
identifying the two layers based on the horizontal plane.
